(19) United States
(12) Patent Application Publication
(10) Pub. No.: US 2019/0159986 A1
(43) Pub. Date: May 30, 2019

(54) Aesthetically Elegant Sexual Lubricants And Massage Gels

(71) Applicant: SXWELL AUSTRALIA PTY. LTD., Richmond (AU)

(72) Inventors: Ji An Chooi, Bayan Lepas, Penang (MY); KC Nguyen, Dothan, AL (US); Beng Sim Chuah, Petaling Jaya (MY); Sek Yan Tan, Seri Kembangan, Selangor (MY); Hanita Binti Mohamad Tahir, Taman Bukit (MY); Victor Virreira, Dothan, AL (US); Chayaporn Pongthanomsak, Suratthani (TH); Maizatul Shuhada Ahmad, Shah Alam, Selangor (MY)

(21) Appl. No.: 16/309,350

(22) PCT Filed: Jun. 22, 2017
(86) PCT No.: PCT/AU2017/000136

§ 371 Date: Dec. 12, 2018

(30) Foreign Application Priority Data
Jun. 30, 2016 (MY) PI2016001234

(51) Int. Cl. A61K 8/34; A61K 8/891; A61Q 19/00

(52) U.S. Cl. CPC A61K 8/345; A61K 8/891; A61K 8/06; A61K 2800/262; A61K 2800/5922; A61Q 19/00

(57) ABSTRACT

Provided among other things is a lubricant formulation comprising by weight: water about 20 to about 45%; propylene glycol about 40 to about 50%; siloxane/emulsifier about 15 to about 30%; and C6 to C12 glycol about 0.5 to about 2%, wherein the formulation is clear to slightly hazy, and wherein additional components are about 5% by weight of the formulation or less.

The present application relates generally to sexual lubricants and massage gels containing water, one or more silicone compounds, and one or more silicone emulsifying agents.

Since the massage gel includes a lubricant function, the sexual lubricant and massage gel embodiments can be jointly referred to as “lubricant formulations.”

The lubricant formulations of the invention provide (i) product clarity, (ii) lack of potential to stain clothing (staining), (iii) high lubricity or (iv) good capacity to maintain lubricity when diluted with water

### SUMMARY

This invention described herein is of lubricant formulations, and method of use thereof. Included is an indexed library comprising features from the independent claims, and methods for using such libraries, substantially as shown in and/or described in connection with at least one of the figures, are disclosed. Various advantages, aspects, and features of the present disclosure, as well as details of illustrated embodiments thereof, will be more fully understood from the following description and drawings. The foregoing summary is not intended, and should not be contemplated, to describe each embodiment or every implementation of the present invention. The Detailed Description and exemplary embodiments therein more particularly exemplify the present invention.

### DETAILED DESCRIPTION

The lubricant formulations of the invention are clear to slightly hazy. By “slightly hazy” it is meant that if a layer 2.5 mm thick is applied to dry skin, an observer maintains a clear sense that he/she can see the skin.

In one embodiment, the invention provides a lubricant formulation comprising by weight:

| | |
|---|---|
| (a) water | about 20 to about 45%; |
| (b) propylene glycol | about 40 to about 50%; |
| (c) siloxane/emulsifier | about 12 to about 30%; and |
| (d) C5 to C12 glycol | about 0.4 to about 2%, |

wherein the formulation is clear to slightly hazy, and wherein additional components are about 5% by weight of the formulation or less. Component (c″) is component (c) less the emulsifier. In embodiments, the C5 to C12 glycol is about 0.6 to about 1.0% of the lubricant formulation.

In an embodiment, the formulation exhibits a coefficient of friction (ASTM G99-05 2010) of about 0.14 or less, such as 0.12 or less, such as about 0.08.

In an embodiment, the formulation can be diluted with water to add 10% (or about 15%, or about 20%) to the original formulation weight and maintain a coefficient of friction of about 0.2 or less.

In embodiments, additional components are about 2% by weight of the formulation or less. In embodiments, additional components are about 1% by weight of the formulation or less. In embodiments, additional components are about 0.5% by weight of the formulation or less. In embodiments, additional components are about 0.2% by weight of the formulation or less.

The invention can provide a packaged condom comprising: a sealed package; within the package, a synthetic polyisoprene condom (substantially polyisoprene, e.g., about 90% of monomer or more); and within the package and substantially coating the condom, a lubricant formulation of the invention.

In embodiments, the lubricant formulation is compatible with condoms, such as synthetic polyisoprene condoms (substantially polyisoprene, e.g., about 90% of monomer or more), natural rubber condom (predominantly natural rubber), and polyurethane condoms (predominantly polyurethane). In embodiments, the lubricant is used in conjunction with the condoms during sexual activity, but not pre-packaged with the condoms. The lubricant formulation can be applied to the condom prior to sexual activity, meaning within about 30 minutes of sexual activity utilizing the condom or during sexual activity to decrease friction at the interface and thus, increase sensitivity for end users.

The invention also can provide a packaged condom comprising: a sealed package; within the package, a polyurethane condom (predominantly polyurethane); and within the package and substantially coating the condom, a lubricant formulation of the invention.

To package a condom dosed with lubricant, for example, metered amounts of the lubricant formulation (such as described above) are applied to the condom. Typically, the lubricant is applied to the rolled condom, such as in the cup formed by the rolling of the condom. If a male condom, for example, this is the opposite to the side on which the penis will be inserted. Typically, the lubricant will migrate over time and distribute itself over the entire exterior and interior surfaces of the condom. The condom can then be sealed, such as in an aluminum foil wrapper.

The siloxane component can include Dimethicone, or Dimethicone and a cyclic siloxane compound, such as octamethylcyclotetrasiloxane, decamethylcyclopentasiloxane, dodecamethylcylcohexasiloxane, or the like.

The emulsifier is present in an amount effective to emulsify in the presence of the components (a), (b) (c″) and (d). In embodiments, it is present in amounts that further assist the acceptance of further water dilution while maintaining good lubricity.

Useful emulsifiers will be recognized by those of ordinary skill in the art. These include esterified (e.g., methyl, ethyl) polyacrylic acid, copolymerized with or substituted with a group having a strong negative charge, such as a charge from a phosphate or sulfate group. For example, the polymer can be copolymerized with 2-methyl-2-[(1-oxo-2-propenyl) amino]-1-propanesulfonic acid.

The C5 to C12 glycol can be a 1,2-diol. In embodiments, more than one C5 to C12 glycol is used. In embodiments, the C5 to C12 glycol is one or more of 1,2-pentanediol, 1,2-hexanediol, 1,2-octanediol, 1,2-decanediol or 1,2-dodecanediol.

The formulations can, for example, be produced with a cold blending procedure. Premix the glycols until uniform. Add the premix into the water base and mix together at their respective ratio until a homogenous mixture is form. Then, the silicone and silicone emulsifying agent is slowly added to the mixture and mixed until uniform. A fragrance for the massage gel can be added at the last step to complete the formulation

In one embodiment, the invention provides a sexual lubricant formulation comprising by weight:

| | |
|---|---|
| water | about 30 to about 45%; |
| propylene glycol | about 40 to about 50%; |
| siloxane/emulsifier | about 12 to about 25%; and |
| C5 to C12 glycol | about 0.4 to about 2%, |

wherein the formulation is clear to slightly hazy, and wherein additional components are about 5% by weight of the formulation or less. In embodiments, the siloxane/emulsifier is about 15 to about 20% by weight of the formulation.

In an embodiment, the sexual lubricant formulation exhibits a coefficient of friction (ASTM G99-05 2010) of about 0.12 or less, such as 0.096 or less, such as about 0.08. Viscosity can be, for example, about 6,000 to about 18,000 cps as measured by an LVT viscometer (e.g., with Brookfield Dial Viscometer, Brookfield Engineering Laboratories, Inc., Middleboro, Mass.).

In an embodiment, the sexual lubricant formulation can be diluted with water to add 10% (or about 15%, or about 20%) to the original formulation weight and maintain a coefficient of friction of about 0.2 or less.

The sexual lubricant formulation can be a light, liquid-like gel, preferably colorless and clear to slight hazy.

In embodiments, the sexual lubricant formulation can include a fragrance (component (e″)), such as a light and pleasant vanilla scent or flavor such as strawberry. The amount of a fragrance or flavor can be, for example, about 0.02 to about 0.2 wt. percent.

In one embodiment, the invention provides a massage gel formulation comprising by weight:

| | |
|---|---|
| (a′) water | about 20 to about 35%; |
| (b′) propylene glycol | about 40 to about 50%; |
| (c′) siloxane/emulsifier | about 20 to about 30%; and |
| (d′) C5 to C12 glycol | about 0.4 to about 2%, |

wherein the formulation is clear to slightly hazy, and wherein additional components are about 5% by weight of the formulation or less. In embodiments, the siloxane/emulsifier is about 22 to about 28% by weight of the formulation.

In an embodiment, the massage gel formulation exhibits a coefficient of friction (ASTM G99-05 2010) of about 0.14 or less, such as 0.12 or less, such as about 0.1.

In an embodiment, the massage gel formulation can be diluted with water to add 10% (or about 15%, or about 20%) to the original formulation weight and maintain a coefficient of friction of about 0.2 or less.

In embodiments, the massage gel formulation can include a fragrance (component (e′)), such as a light and pleasant vanilla scent, or flavor such as strawberry. The amount of a fragrance or flavor can be, for example, about 0.02 to about 0.2 wt. percent.

In embodiments, the viscosity of the massage gel is selected to be appropriate for message, such as to provide a better gliding effect and composition body that helps to enhance the massaging effect. For example, the viscosity can be about 40,000 to about 100,000 cps, as measured by an LVT viscometer. In embodiments, components (a′) through (e′) comprise 99% or more by weight (or 99.5% or more, or 99.7% or more) of the massage gel composition, while providing such viscosity.

In embodiments, the lubricant formulations of the invention spreads easily on the skin and also provides a long play time with silky smooth finish.

In embodiments, the lubricant formulations of the invention maintains the silicone lubricant properties of good lubricity and at the same time is stain-free, in contrast to other silicone lubricants.

Stain testing can be conducted for example on cotton, polyester, satin and synthetic silk. Grid lines were drawn on the fabrics, such as:

The grid squares can by 10 by 12 mm. Apply 3 g of selected fluid on fabric surface within a grid. One grid can be left free as a control.

Sketch the stain mark on the fabric, then leave for 24 hours. The sheet of fabric is cleaned without detergent in a typical household washing machine using the guide below:

| Washing machine | Polyester & Cotton | Satin & Synthetic silk |
|---|---|---|
| Water level | Mini | Mini |
| Wash/rinse temp | Cold/cold | Cold/cold |
| Fabric selection | Normal | Gentle care |
| Washing cycle | Daily wash (55 min) | Daily wash (55 min) |

The fabric is allowed to dry at room temperature. Using a sexual lubricant composition and a massage gel composition of the invention, no staining was seen.

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more. If there are two ranges mentioned, such as about 1 to 10 and about 2 to 5, those of skill will recognize that the implied ranges of 1 to 5 and 2 to 10 are within the invention.

Where a sentence states that its subject is found in embodiments, or in certain embodiments, or in the like, it is applicable to any embodiment in which the subject matter can be logically applied.

The invention can be further described with respect to the following embodiments:

### Embodiment 1

A lubricant formulation comprising by weight:

| | |
|---|---|
| water | about 20 to about 45%; |
| propylene glycol | about 40 to about 50%; |
| siloxane/emulsifier | about 15 to about 30%; and |
| C5 to C12 glycol | about 0.5 to about 2%, |

wherein the formulation is clear to slightly hazy, and wherein additional components are about 5% by weight of the formulation or less.

### Embodiment 2

The lubricant embodiment of this section, wherein the coefficient of friction exhibited (ASTM G99-05 2010) is about 0.14 or less.

### Embodiment 3

The lubricant embodiment of this section, wherein the formulation can be diluted with water to add 10% to the original formulation weight and maintain a coefficient of friction of about 0.2 or less.

### Embodiment 4

A method of using a lubricant embodiment of this section, comprising applying the formulation to skin or mucosa to make it more lubricated, and conducting sexual or massage activity that is facilitated by the lubricated skin or mucosa.

### Embodiment 5

The lubricant embodiment of this section, comprising a fragrance or flavor.

### Embodiment 6

A method of using a lubricant embodiment of this section, comprising applying the formulation to a condom prior to sexual activity to enhance sensitivity.

### Embodiment 7

The method of Embodiment 6, wherein the condom is synthetic polyisoprene, natural rubber, or polyurethane.

### Embodiment 8

The lubricant formulation of claim 1 formulated as a sexual lubricant and comprising:

| | |
|---|---|
| water | about 30 to about 45%; |
| propylene glycol | about 40 to about 50%; |
| siloxane/emulsifier | about 15 to about 25%; and |
| C5 to C12 glycol | about 0.5 to about 2%. |

### Embodiment 9

A method of using a lubricant embodiment of this section, comprising applying the formulation to skin or mucosa to make it more lubricated, and conducting sexual activity that is facilitated by the lubricated skin or mucosa.

### Embodiment 10

A method of using a lubricant embodiment of this section, comprising applying the formulation to condom prior to sexual activity to enhance sensitivity.

### Embodiment 11

The method of claim 10, wherein the condom is synthetic polyisoprene, natural rubber, or polyurethane.

### Embodiment 12

The lubricant formulation of Embodiment 1 formulated as a massage gel and comprising:

| | |
|---|---|
| water | about 20 to about 35%; |
| propylene glycol | about 40 to about 50%; |
| siloxane/emulsifier | about 20 to about 30%; and |
| C5 to C12 glycol | about 0.5 to about 2%. |

### Embodiment 13

A method of using a lubricant embodiment of this section, comprising applying the formulation to skin to make it more lubricated, and conducting massage activity that is facilitated by the lubricated skin.

This invention described herein is of a lubricating formulation and methods of using or forming the same. Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

1. A lubricant formulation comprising by weight:
(a) about 20 to about 45% water;
(b) about 40 to about 50% propylene glycol;
(c) about 5-12 to about 30% siloxane and/or emulsifier; and
(d) about 0.4 to about 2% C₅ to C₁₂ glycol;
wherein the formulation is clear to slightly hazy, and wherein additional components are about 5% by weight of the formulation or less.

2. The lubricant formulation of claim 1 formulated as a sexual lubricant and comprising:
(a) about 30 to about 45% of the water;
(b) about 40 to about 50% of the propylene glycol;
(c) about 5-12 to about 25% of the siloxane and/or emulsifier; and
(d) about 0.4 to about 2% of the C₅ to C₁₂ glycol.

3. The lubricant formulation of claim 1 formulated as a massage gel and comprising:
(a) about 20 to about 35% of the water;
(b) about 40 to about 50% of the propylene glycol;
(c) about 20 to about 30% of the siloxane and/or emulsifier; and
(d) about 0.4 to about 2% of the C₅ to C₁₂ glycol.

4. The lubricant formulation of claim 1, comprising a coefficient of friction of about 0.14 or less.

5. The lubricant formulation of claim 1, wherein upon dilution of the lubricant formulation with water to add 10% to an original formulation weight, a coefficient of friction of about 0.2 or less is maintained.

6. The lubricant formulation of claim 1, further comprising a fragrance or a flavor.

7. A method of using a lubricant formulation comprising (a) about 20 to about 45% water, (b) about 40 to about 50% propylene glycol, (c) about 12 to about 30% siloxane and/or emulsifier, and (d) about 0.4 to about 2% C₅ to C₁₂ glycol, the method comprising:
applying the lubricant formulation to skin or mucosa to create lubricated skin or mucosa, and
conducting sexual or massage activity that is facilitated by the lubricated skin or mucosa.

8. A method of using a lubricant formulation comprising (a) about 20 to about 45% water, (b) about 40 to about 50% propylene glycol, (c) about 12 to about 30% siloxane and/or emulsifier, and (d) about 0.4 to about 2% C₅ to C₁₂ glycol, the method comprising applying the lubricant formulation to a condom prior to sexual activity to enhance sensitivity.

9. The method of claim 8, wherein the condom is synthetic polyisoprene, natural rubber, or polyurethane.

10. The method of claim 7, wherein the lubricant formulation comprises (a) about 30 to about 45% of the water, (b) about 40 to about 50% of the propylene glycol, (c) about 12 to about 25% of the siloxane and/or emulsifier, and (d) about 0.4 to about 2% of the C₅ to C₁₂ glycol, the method comprising conducting sexual activity.

11. The method of claim 7, wherein the lubricant formulation comprises (a) about 20 to about 35% of the water, (b) about 40 to about 50% of the propylene glycol, (c) about 20 to about 30% of the siloxane and/or emulsifier, and (d) about 0.4 to about 2% C₅ to C₁₂ of the glycol, the method comprising conducting massage activity.

12. The lubricant formulation of claim 1, wherein the C₅ to C₁₂ glycol is, 2-pentanediol, 1,2-hexanediol, 1,2-octanediol, 1,2-decanediol or 1,2-dodecanediol, or combinations thereof.

13. The lubricant formulation of claim 2, wherein the C₅ to C₁₂ glycol is, 2-pentanediol, 1,2-hexanediol, 1,2-octanediol, 1,2-decanediol or 1,2-dodecanediol, or combinations thereof.

14. The lubricant formulation of claim 3, wherein the C₅ to C₁₂ glycol is, 2-pentanediol, 1,2-hexanediol, 1,2-octanediol, 1,2-decanediol or 1,2-dodecanediol, or combinations thereof.
